(19) United States
(12) Patent Application Publication
(10) Pub. No.: US 2025/0384599 A1
(43) Pub. Date: Dec. 18, 2025

(54) X-RAY CT APPARATUS, MODEL GENERATION SYSTEM, MODEL GENERATION METHOD, AND INFORMATION PROCESSING APPARATUS

(71) Applicant: CANON MEDICAL SYSTEMS CORPORATION, Otawara-shi (JP)

(72) Inventors: Sho SASAKI, Ota (JP); Asateru KIMURA, Yokohama (JP); Takuo NEGISHI, Nasushiobara (JP)

(73) Assignee: CANON MEDICAL SYSTEMS CORPORATION, Otawara-shi (JP)

(21) Appl. No.: 19/215,877

(22) Filed: May 22, 2025

(30) Foreign Application Priority Data
Jun. 14, 2024 (JP) 2024-096751

(51) Int. Cl. G06T 11/00; G16H 15/00; G16H 40/20

(52) U.S. Cl. CPC G06T 11/006; G16H 15/00; G16H 40/20

(57) ABSTRACT

An X-ray CT apparatus according to an embodiment includes an X-ray tube, an X-ray detector, a processor, and a memory. The memory stores a global model to be used in federated learning. The processor generates CT image data by executing reconstruction processing on detection data of X-rays. The processor transmits, to a client, the global model and control information controlling execution of a trainer at the client. The processor acquires, from the client, a local model generated by training of the global model with training data by the trainer under control of the control information. The processor updates the control information in accordance with a training log of the client. A model generation system according to another embodiment includes a central server and a client. Still another embodiment discloses a model generation method implemented by a client and a central server.

### CROSS-REFERENCE TO RELATED APPLICATIONS

This application is based upon and claims the benefit of priority from Japanese Patent Application No. 2024-096751, filed on Jun. 14, 2024; the entire contents of all of which are incorporated herein by reference.

### FIELD

Embodiments disclosed herein relate generally to an X-ray CT apparatus, a model generation system, a model generation method, and an information processing apparatus.

### BACKGROUND

Federated learning (FL) has been known as a method for training of an artificial intelligence (AI) model. In the federated learning, a central server integrates local models, which have been trained at plural clients, into a global model. The training method like this enables additional training of the global model without transmitting training data from the clients to the central server. Therefore, it is useful for efficiently performing additional training while reducing leakage risk in fields where personal information is included in the training data.

However, in the federated learning, training is executed at more than one client, so that some clients may execute training with data that are inappropriate.

### BRIEF DESCRIPTION OF THE DRAWINGS

FIG. 1 is a diagram illustrating an example of a configuration of a model generation system according to a first embodiment;

FIG. 2 is a block diagram illustrating an example of a configuration of a central server according to the first embodiment;

FIG. 3 is a block diagram illustrating an example of a configuration of a client according to the first embodiment;

FIG. 4 is a sequence diagram illustrating an example of a procedure of processing for additional training according to the first embodiment;

FIG. 5 is a block diagram illustrating an example of a configuration of a central server and a client, according to a second embodiment;

FIG. 6 is a sequence diagram illustrating an example of a procedure of processing for additional training according to the second embodiment;

FIG. 7 is a block diagram illustrating an example of a configuration of a central server and a client, according to a third embodiment;

FIG. 8 is a sequence diagram illustrating an example of a procedure of processing for additional training according to the third embodiment;

FIG. 9 is a diagram illustrating an example of a configuration of a model generation system according to a fourth embodiment;

FIG. 10 is a diagram illustrating an example of a configuration of an X-ray CT apparatus according to the fourth embodiment;

FIG. 11 is a diagram illustrating an example of a configuration of a model generation system according to a fifth embodiment; and

FIG. 12 is a diagram illustrating an example of a configuration of a model generation system according to a sixth embodiment.

### DETAILED DESCRIPTION

Embodiments of an X-ray CT apparatus, a model generation system, a model generation method, and an information processing apparatus will be described in detail hereinafter, while reference is made to the drawings.

An X-ray CT apparatus according to an embodiment includes an X-ray tube, an X-ray detector, at least one memory, and at least one piece of processing circuitry. The X-ray tube is configured to emit X-rays to a subject. The X-ray detector is configured to detect X-rays emitted from the X-ray tube. The memory is configured to store a global model to be used in federated learning. The processing circuitry is connected to the memory. The processing circuitry is configured to generate CT image data by executing reconstruction processing on detection data of the X-rays detected by the X-ray detector. The processing circuitry is configured to transmit, to a client, the global model and control information controlling execution of a trainer at the client. The processing circuitry is configured to acquire, from the client, a local model generated by training of the global model with training data by the trainer under control of the control information. The processing circuitry is configured to update the control information in accordance with a training log of the client.

A model generation system according to an embodiment includes a central server and a client. The client is capable of executing a trainer. The central server is capable of providing the client with a global model to be used in federated learning. The client is configured to apply, to the trainer, the global model acquired from the central server. The client is configured to generate a local model by inputting training data to the trainer. The client is configured to provide the central server with the local model. Execution of the trainer at the client is controlled by control information assigned to the trainer. The central server is configured to execute control to enable the control information to be changed with a training log of the client.

### First Embodiment

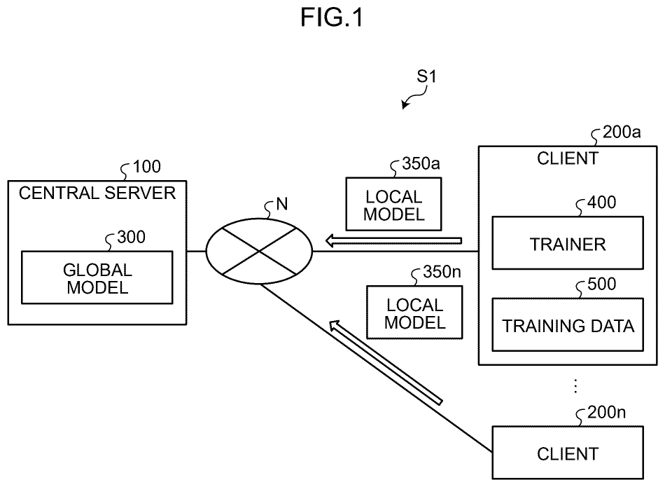

FIG. 1 is a diagram illustrating an example of a configuration of a model generation system S1 according to a first embodiment. As illustrated in FIG. 1, the model generation system S1 includes a central server 100 and plural clients 200a to 200n.

The model generation system S1 is a system that executes machine learning by means of federated learning (FL). Federated learning refers to a learning method, in which the central server 100 integrates plural local models 350a to 350n that have been trained at the plural clients 200a to 200n, into a global model 300. Repetition of training of the local models 350a to 350n and integration of these local models 350a to 350n into the global model 300 enables additional training of the global model 300. Moreover, the unit of repetition, in which the global model 300 is repeatedly updated by use of the local models 350a to 350n in the federated learning is called “round”.

Federated learning is also called associative learning or collaborative learning. The global model 300 is also called a parent model and the local models 350a to 350n are also called child models. In a case where the plural local models 350a to 350n are not to be distinguished from one another, they will hereinafter be simply referred to as local models 350. Moreover, in a case where the individual clients 200a to 200n are not to be particularly distinguished from one another, they will simply be referred to as clients 200. The clients 200 are each an example of an information processing apparatus according to the present embodiment.

A model according to the present embodiment is a set of parameters defining a relation between data inputs and outputs. More specifically, the global model 300 is an artificial intelligence (AI) model that has been trained. The local models 350 are models resulting from additional training of the global model 300 by the clients 200. The kind of training that the global model 300 and the local models 350 are subjected to is, for example, machine learning for linear regression models and deep neural networks. The kind of training is not limited to these examples, and any publicly known technique may be adopted.

The central server 100 and the clients 200 are connected to be able to communicate with each other via a network N, such as the Internet, for example.

The central server 100 is a computer that is capable of providing the clients 200 with the global model 300 to be used in the federated learning. The central server 100 is an example of an information processing apparatus according to the present embodiment. The central server 100 may be called a first information processing apparatus, and a client 200 may be called a second information processing apparatus. Either one of the central server 100 or a client 200 may be called an information processing apparatus and the other one may be called the other information processing apparatus.

The clients 200a to 200n are each capable of executing a trainer 400 and each apply the global model 300 acquired from the central server 100 to the trainer 400.

More specifically, the clients 200 each generate a local model by providing the trainer 400 with training data 500. The clients 200 are each capable of providing the central server 100 with the local model.

The training data 500 include, for example, medical information. In a more specific example, the training data 500 is personal medical data recorded as, for example, a personal health record (PHR). The data format of the training data 500 is not particularly limited, but for example, the training data 500 may include both or any one of text data and image data. In the present embodiment, one client 200 corresponds to one patient. A client 200 may be a computer carried by a patient, such as a personal computer (PC), a tablet terminal, or a smartphone. The training data 500 may be stored in, for example, a server of an enterprise that provides PHRs. In this case, the client 200 may download the training data 500 from, for example, the server of the enterprise of the PHRs. The training data 500 may be referred to as learning data.

The training data 500 is a data set in which data corresponding to input data for inference and true data corresponding to the data have been correlated with each other. The true data is also called a label. In one example, the training data 500 may be a data set obtained by correlating a result of a physical examination or various tests on a patient with a result of diagnosis on the patient. In the present embodiment, one data set will be referred to as one set of training data 500. The content of the training data 500 is not limited to this example.

The trainer 400 generates the local model 350 by inputting the training data 500 to the global model 300 and performing training of the global model 300. The trainer 400 is, for example, an application program capable of executing machine learning of a linear regression model, a deep neural network, or the like, as described above.

Moreover, a constraint related to training is imposed on each of the clients 200a to 200n by the central server 100.

The constraint is, for example, an upper limit number of executions or an executable time period for the trainer 400 in each of the clients 200a to 200n. The central server 100 transmits control information for controlling the trainer 400 based on the constraint to each of the clients 200a to 200n. Therefore, execution of the trainer 400 at each of the clients 200a to 200n is controlled by the constraint assigned to the trainer 400.

The upper limit number of executions for the trainer 400 is an upper limit number of times of training for training of the global model 300 by the trainer 400 in one round. The trainer 400 generates one local model 350 in each round. One set of training data 500 is input to the global model 300 every time the trainer 400 is executed once. Thus, the upper limit number of executions for the trainer 400 also refers to an upper limit number of sets of training data 500 to be used in training of the global model 300 for one local model 350 to be generated.

The executable time period refers to an upper limit of a processing time period for training of the global model 300 by the trainer 400 in one round. In other words, the executable time period is an upper limit of a time period during which training processing for training of the global model 300 by the trainer 400 is able to be executed for generating one local model 350.

The constraints placed on the clients 200a to 200n may be the same or different from one another. In one example, the central server 100 may execute control such that the constraints placed on the clients 200a to 200n are able to change with training logs of training at the respective clients 200a to 200n.

By placing such a constraint on a client 200, the central server 100 constrains the client 200 from performing training of a quantity equal to or larger than that requested by the central server 100. Such a restriction enables reduction of influence of contamination of the global model 300 by a malicious attacker on the global model 300.

It is now supposed that, for example, a person (attacker) who intends to contaminate the global model 300 is included in users of the plural clients 200a to 200n. Examples of such an attack include a poisoning attack and a backdoor attack. The poisoning attack is an attack performed such that the training data 500 are contaminated and thereby errors are added in the local model 350 that has been trained, and the global model 300, into which this local model 350 has been integrated, is thereby induced to output an incorrect inference result in the inference phase. The backdoor attack is an attack, in which the local model 350 is trained to output an incorrect inference result only for any input including a specific trigger and the global model 300, into which this local model 350 has been integrated, is similarly induced to output an incorrect inference result only for any input including a specific trigger. In a case where such an attack is intended, an attacker typically attempts to increase influence on the global model 300 by executing training using a large amount of corrupted data. However, the constraints constrain the clients 200 from performing training of quantities equal to or larger than those requested by the central server 100, and the influence that the individual clients 200 have on the global model 300 is thus limited.

It is now supposed that the total number of clients 200 included in the model generation system S1 is 1,000 and, among these 1,000 clients 200, the number of clients 200 manipulated by a user who intends to attack the global model 300 is 100. In this case, if the upper limit numbers of executions for the trainers 400 in the clients 200 in one round are prescribed to be the same by the constraints, contaminated data is prevented from accounting for more than 10% of all the training data.

In order for attackers to execute training with large amounts of corrupted data under these constraints, the attackers need to execute the training by dividing the training in plural rounds. Therefore, time and effort taken by the attackers are increased and motivation of the attackers to attack can thus be expected to be discouraged.

Moreover, even if an operator has no intention to attack, unintended mistraining may be executed at a client 200. For example, data not planned to be used in additional training or data not suitable for additional training may be used as training data 500 by a malfunction at a client 200 or incorrect operation by an operator. Specifically, an operator may input a wrong label, or data still being generated may be input by mistake. Even in such a case, the constraint constrains each client 200 from executing training of a quantity equal to or larger than that requested by the central server 100, and influence that a local model 350 that has been subjected to mistraining has on the global model 300 is thus able to be reduced.

The number of clients 200 included in the model generation system S1 is not particularly limited. An increase in the amount of data used in training at each client 200 as described above may lead to increased chances of malicious contamination or mistraining. Therefore, in a case where there is a need to increase the number of sets of training data 500 required for additional training of the global model 300, increasing the number of clients 200 is more desirable than increasing the upper limit number of executions per client 200.

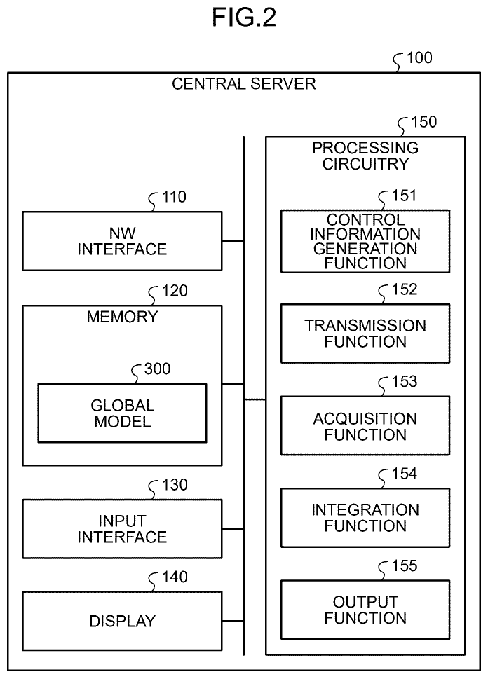

Details of a configuration of the central server 100 will be described next. FIG. 2 is a block diagram illustrating an example of the configuration of the central server 100 according to the first embodiment.

As illustrated in FIG. 2, the central server 100 includes a network (NW) interface 110, a memory 120, an input interface 130, a display 140, and processing circuitry 150.

The NW interface 110 has been connected to the processing circuitry 150 and controls transmission and communication of various kinds of data between the central server 100 and the plural clients 200a to 200n. The NW interface 110 is implemented by a network card, a network adapter, or a network interface controller (NIC), for example.

The memory 120 stores various kinds of information to be used by the processing circuitry 150, beforehand. Moreover, the memory 120 stores the global model 300 and various programs. The memory 120 is, for example, a nonvolatile storage device, such as a hard disk drive (HDD), a solid state drive (SSD), or an integrated circuit storage device, which stores various kinds of information. The memory 120 may be other than an HDD or an SSD, and may be, for example, a drive device that reads and writes various kinds of information from and into: a portable storage medium, such as a compact disc (CD), a digital versatile disc (DVD), or a flash memory; or a semiconductor memory element, such as a random access memory (RAM). Moreover, the memory 120 stores the global model 300, for example.

The input interface 130 is implemented by any of: a mouse; a keyboard; a pen tablet, which is a combination of a touch pen and a tablet that receive operation by a user; a trackball; switch buttons; a touch pad, through which input operation is performed by contact with an operating surface; a touch screen having a display screen and a touch pad integrated together; a non-contact input circuit using an optical sensor; and a voice input circuit. The input interface 130 may include plural devices that receive operation by a user. The input interface 130 has been connected to the processing circuitry 150, converts input operation received from a user into an electric signal, and outputs the electric signal to the processing circuitry 150. According to the present specification, the input interface 130 is not necessarily an input interface including physical operating parts, such as a mouse and a keyboard. Examples of the input interface 130 include electric signal processing circuitry that receives an electric signal corresponding to input operation from an external input device provided separately from the apparatus and outputs this electric signal to the processing circuitry 150.

Under control of the processing circuitry 150, the display 140 displays various kinds of information. For example, the display 140 may output a graphical user interface (GUI) for receiving various kinds of operation from a user. Specifically, the display 140 is, for example, a liquid crystal display or a cathode ray tube (CRT) display. The input interface 130 and the display 140 may be integrated with each other. In one example, the input interface 130 and the display 140 may be implemented by a touch panel.

The processing circuitry 150 is a processor that implements functions corresponding to programs by reading and executing the programs from the memory 120. The processing circuitry 150 according to the present embodiment includes a control information generation function 151, a transmission function 152, an acquisition function 153, an integration function 154, and an output function 155. The control information generation function 151 is an example of a control information generation unit and a control information update unit. The transmission function 152 is an example of a transmission unit. The acquisition function 153 is an example of an acquisition unit. The integration function 154 is an example of an integration unit. The output function 155 is an example of an output unit.

Processing functions of the control information generation function 151, the transmission function 152, the acquisition function 153, the integration function 154, and the output function 155, which are elements of the processing circuitry 150, have been stored in the form of programs that are able to be executed by a computer, in the memory 120. The processing circuitry 150 is a processor. The processing circuitry 150 implements the functions corresponding to the programs by reading and executing the programs from the memory 120. In other words, the processing circuitry 150 that has read the programs has the functions illustrated in the processing circuitry 150 in FIG. 2. The processing functions implemented by the control information generation function 151, the transmission function 152, the acquisition function 153, the integration function 154, and the output function 155 have been described as being implemented by a single processor by reference to FIG. 2, but the processing circuitry 150 may include a combination of plural independent processors and the functions may be implemented by the processors executing the programs. It has been described by reference to FIG. 2 that the single memory 120 stores the programs corresponding to the processing functions, but plural memories may be arranged in a distributed manner and the processing circuitry 150 may be configured to read the corresponding programs from the individual memories.

The control information generation function 151 generates control information defining the constraints to be placed on the clients 200. The control information generation function 151 generates, based on operation by an administrator of the central server 100, control information defining upper limit numbers of executions or executable time periods for the trainers 400 in the clients 200.

Moreover, the control information generation function 151 may execute control to enable the control information to be changed with training logs of training at the clients 200. For example, the control information generation function 151 may increase the upper limit number of executions or the executable time period for the trainer 400 of any client 200 that has gone through a prescribed number of rounds or more.

The transmission function 152 transmits permission for execution of additional training, the global model 300, and the control information, to the clients 200, via the NW interface 110 and the network N.

The acquisition function 153 acquires a start request for additional training from the clients 200. Moreover, the acquisition function 153 acquires the local models 350 from the clients 200.

The integration function 154 integrates the local models 350 acquired from the clients 200 by the acquisition function 153, into the global model 300. The global model 300, into which the local models 350 have been integrated, is also called a new global model 300 or the global model 300 that has been additionally trained.

The output function 155 controls the display 140 to cause the display 140 to display various kinds of information. The output function 155 may cause the display 140 to display, for example, information representing that integration of the local models 350 into the global model 300 has been completed.

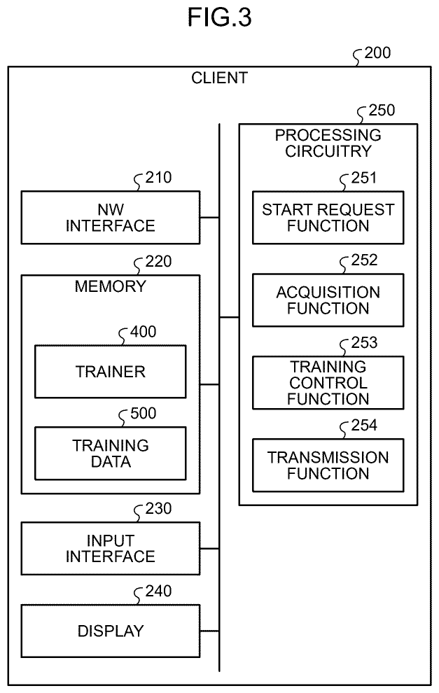

Details of a configuration of a client 200 will be described next. FIG. 3 is a block diagram illustrating an example of the configuration of the client 200 according to the first embodiment.

As illustrated in FIG. 3, the client 200 includes an NW interface 210, a memory 220, an input interface 230, a display 240, and processing circuitry 250.

The NW interface 210 has been connected to the processing circuitry 250 and controls transmission and communication of various kinds of data between the client 200 and the central server 100.

The memory 220 stores various kinds of information and various computer programs to be used by the processing circuitry 250, beforehand. For example, the memory 220 stores training data 500. Moreover, the memory 220 stores an application program for a trainer 400. The memory 220 may be: a nonvolatile storage device, such as an HDD, an SSD, or an integrated circuit storage device; or a drive device that reads and writes various kinds of information from and into a portable storage medium, such as a CD, a DVD, or a flash memory, or a semiconductor memory element, such as a RAM. Moreover, the memory 220 stores, for example, the trainer 400 and a local model 350.

The input interface 230 may be implemented by signal processing circuitry that outputs, to the processing circuitry 250, an electric signal received through any of a mouse, a keyboard, a pen tablet, a trackball, switch buttons, a touch pad, a touch screen, a non-contact input circuit, a voice input circuit, an external input device, etc. The input interface 230 may include plural devices. The input interface 230 has been connected to the processing circuitry 250, converts input operation received from a user into an electric signal, and outputs the electric signal to the processing circuitry 250.

Under control of the processing circuitry 250, the display 240 displays various kinds of information.

The processing circuitry 250 is a processor that implements functions corresponding to programs by reading and executing the programs from the memory 220. The processing circuitry 250 according to the present embodiment includes a start request function 251, an acquisition function 252, a training control function 253, and a transmission function 254. The start request function 251 is an example of a start request unit. The acquisition function 252 is an example of an acquisition unit. The training control function 253 is an example of a training control unit. The transmission function 254 is an example of a transmission unit.

Processing functions of the start request function 251, the acquisition function 252, the training control function 253, and the transmission function 254, which are elements of the processing circuitry 250, have been stored in the memory 220 in the form of computer programs that are able to be executed by a computer. The processing circuitry 250 is a processor. The processing circuitry 250 implements the functions corresponding to the programs by reading and executing the programs from the memory 220. In other words, the processing circuitry 250 that has read the programs has the functions illustrated in the processing circuitry 250 in FIG. 3. The processing functions implemented by the start request function 251, the acquisition function 252, the training control function 253, and the transmission function 254 have been described as being implemented by a single processor by reference to FIG. 3, but the processing circuitry 250 may include a combination of plural independent processors and the functions may be implemented by the processors executing the programs. Moreover, it has been described by reference to FIG. 3 that the single memory 220 stores the programs corresponding to the processing functions, but plural memories may be arranged in a distributed manner and the processing circuitry 250 may be configured to read the corresponding programs from the individual memories.

In a case where a state where additional training is able to be executed at a client 200 has been reached, the start request function 251 transmits a start request for additional training, to the central server 100. This start request is newly transmitted for each round. For example, in a case where training data 500 required for additional training in a subsequent round have been stored in the memory 220 after completion of additional training of a previous round, the start request function 251 may transmit a start request for additional training to the central server 100.

It is now supposed that, for example, the training data 500 required for the additional training is data about a result of a physical examination on a patient corresponding to the client 200. In this case, when the data about the physical examination on the patient have been stored in the memory 220, the start request function 251 may transmit a start request for additional training, to the central server 100.

The acquisition function 252 acquires permission for execution of additional training, the global model 300, and the control information, from the central server 100, via the NW interface 110 and the network N.

In a case where the training control function 253 has received permission for execution of additional training, the training control function 253 activates the trainer 400 to cause the trainer 400 to execute training based on the control information. Under the constraint defined by the control information, the trainer 400 trains the global model 300 acquired from the central server 100, using the training data 500. In FIG. 3, the trainer 400 and the training control function 253 are illustrated separately from each other, but the trainer 400 may function as the training control function 253.

The transmission function 254 transmits the local model 350 generated by additional training of the global model 300 by the trainer 400, to the central server 100.

An example where the “processor” reads the programs corresponding to the functions from the memory and executes the programs has been described above with respect to the central server 100 and the clients 200, but the embodiment is not limited to this example. The term, “processor”, means, for example, any of circuits including a central processing unit (CPU), a graphics processing unit (GPU), an application specific integrated circuit (ASIC), and programmable logic devices (for example, a simple programmable logic device (SPLD), a complex programmable logic device (CPLD), and a field programmable gate array (FPGA)). In a case where the processor is a CPU, for example, the processor implements the functions by reading and executing the programs stored in the memory. In a case where the processor is an ASIC, instead of the programs being stored in the memory 120 or 220, the functions are directly incorporated, as logic circuits, into circuitry of the processor. Each of the processors according to the embodiment is not necessarily configured as a single circuit, and plural independent circuits may be combined together to be configured as a single processor to implement the functions. Moreover, plural elements in FIG. 2 and FIG. 3 may be integrated into a single processor for their functions to be implemented.

A procedure of processing for additional training executed by the model generation system S1 according to the present embodiment configured as described above will be described next.

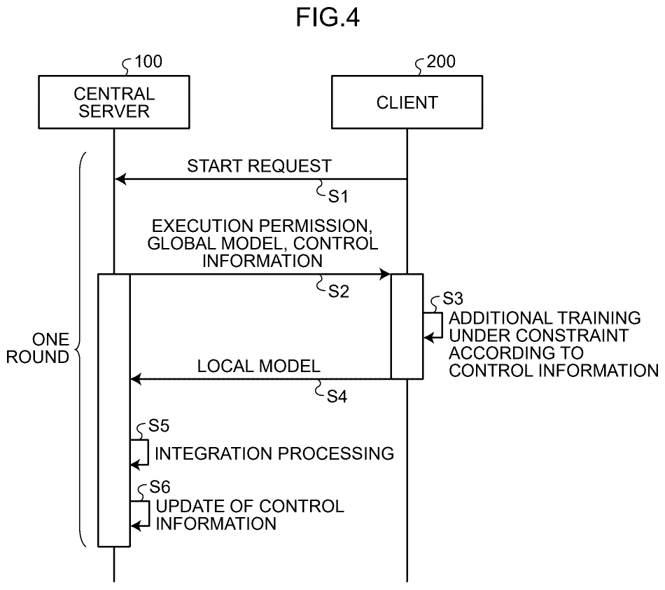

FIG. 4 is a sequence diagram illustrating an example of a procedure of processing for additional training according to the first embodiment. As a premise of this processing illustrated in FIG. 4, it is supposed that the control information generation function 151 of the central server 100 has already generated control information defining a constraint to be placed on the trainer 400 at a client 200.

Firstly, the start request function 251 at the client 200 transmits a start request to the central server 100 (S1).

Upon receipt of the start request from the client 200, the transmission function 152 of the central server 100 transmits permission for execution of additional training, the global model 300, and the control information, to the client 200 (S2). In a case where the transmission function 152 has received the start request, the transmission function 152 may refrain from transmitting permission for execution of additional training until the central server 100 is prepared for the additional training. For example, in a second or later round, in a case where integration of a local model 350 acquired in a previous round into the global model 300 has been completed, the transmission function 152 transmits the permission for execution of additional training. Moreover, for example, the central server 100 may receive a start request for additional training from the client 200 only during prescribed hours of day.

The acquisition function 252 at the client 200 acquires the permission for execution of additional training, the global model 300, and the control information, which have been transmitted from the central server 100. The training control function 253 at the client 200 activates the trainer 400 and provides the trainer 400 with training data 500 to cause the trainer 400 to execute training based on the control information. Under the constraint defined by the control information, the trainer 400 trains the global model 300 acquired from the central server 100 by using the training data 500 (S3). For example, in a case where the constraint is “the upper limit number of executions=1”, the trainer 400 executes additional training of the global model 300 with one set of training data 500. By the trainer 400 executing the additional training, a local model 350 is generated. One set of training data 500 may refer to a data group that is a group of plural data files, without being limited to a single data file. That is, batch processing executed by grouping plural sets of data together into one group may be regarded as handling of one set of training data 500. However, in terms of restraining the client 200 from executing training of a quantity equal to or larger than that requested by the central server 100, an upper limit value for the amount of data handled per set of batch processing may be set by the constraint.

The transmission function 254 at the client 200 then transmits the local model 350 generated by the trainer 400, to the central server 100 (S4).

The acquisition function 153 of the central server 100 acquires the local model 350 from the client 200. The integration function 154 of the central server 100 then executes integration processing of integrating the acquired local model 350 into the global model 300 (S5). The global model 300 is thereby updated. After the integration processing at S5 is completed, the output function 155 of the central server 100 may cause the display 140 to display, for example, information representing that the integration of the local model 350 into the global model 300 has been completed.

The control information generation function 151 of the central server 100 updates the control information in accordance with a training log of the client 200 (S6). For example, the control information generation function 151 increases the upper limit number of executions or the executable time period for the trainer 400 in a case where the number of rounds of additional training executed by the client 200 has exceeded a prescribed number of rounds. The updated control information is transmitted from the central server 100 to the client 200 in a subsequent round. The processing at S6 is not indispensable. The processing in this sequence diagram is then ended.

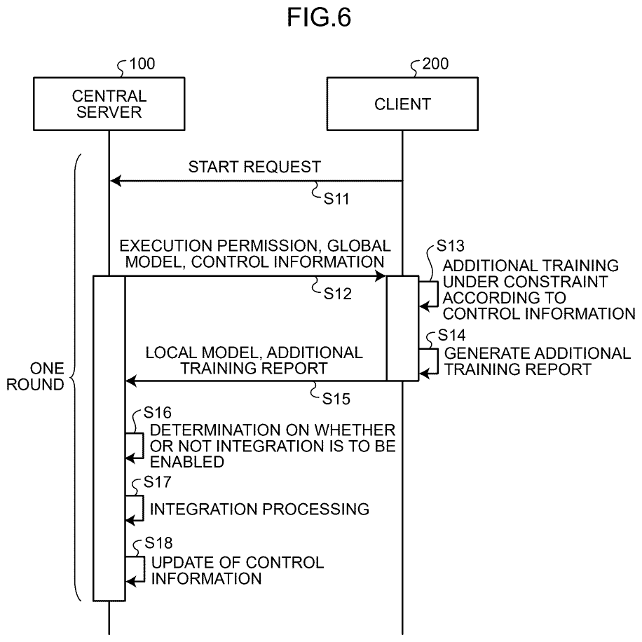

The processing at S1 to S6 in FIG. 6 corresponds to one round of additional training. By repeatedly executing a plural number of rounds, the model generation system S1 implements federated learning of the global model 300.

A procedure of processing between the central server 100 and one client 200 has been described above with reference to FIG. 4, but the central server 100 executes similar processing with each of the plural clients 200a to 200n.

As described above, the model generation system S1 according to the present embodiment includes the central server 100 and the client 200. The central server 100 is capable of providing the client 200 with the global model 300 used in federated learning. The client 200 applies the global model 300 to the trainer 400 and provides the trainer 400 with the training data 500 to cause the trainer 400 to generate the local model 350. The client 200 then provides the central server 100 with the local model 350 generated by the trainer 400. In the model generation system S1 according to the present embodiment, execution of the trainer 400 at the client 200 is controlled by the control information assigned to the trainer 400. Moreover, the central server 100 executes control to enable the control information to be changed with the training log of the client 200. Therefore, the model generation system S1 according to the present embodiment enables control of additional training by the client 200 and quality of the additional training to be maintained in the federated learning.

Moreover, the control information according to the embodiment is information defining the upper limit number of executions or the executable time period for the trainer 400. By placing such a constraint on the client 200, the central server 100 restrains the client 200 from executing training of a quantity equal to or larger than that requested by the central server 100, and influence of contamination by a malicious attacker and unintended mistraining is thereby able to be reduced.

Moreover, in the present embodiment, the training data 500 include medical information on a patient, and the clients 200 have a one-to-one correspondence with patients. Although the medical information typically includes personal information, the federated learning enables the additional training without transmission of the medical information itself to the central server 100 and thus enables privacy of the patients to be protected. In a case where the clients 200 correspond to the patients one-to-one, the number of sets of training data 500 that a single client 200 can provide is typically small. Therefore, for training using medical information on patients, there are not many drawbacks in applying the technique of limiting the number of times of training per client 200 through the constraint, and the technique for the additional training according to the present embodiment is thus suitable.

### Second Embodiment

In a second embodiment, a client 200 generates a report related to additional training executed and provides the report to a central server 100. Based on the report, the central server 100 determines whether or not a local model 350 generated by the client 200 is to be integrated into a global model 300.

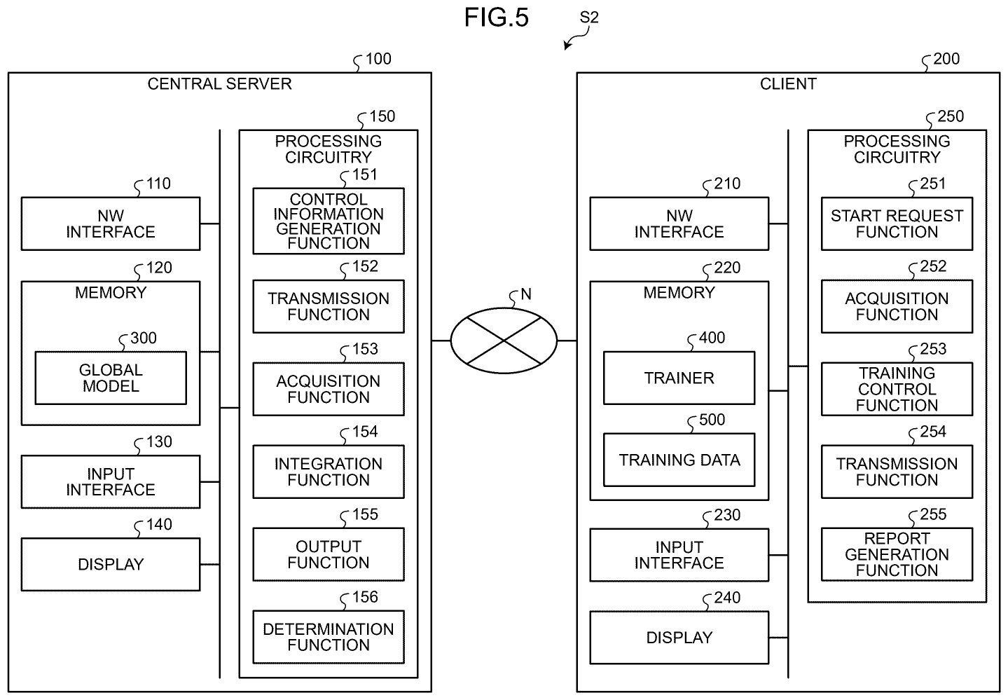

FIG. 5 is a block diagram illustrating an example of a configuration of the central server 100 and the client 200, according to the second embodiment. A model generation system S2 according to the present embodiment includes the central server 100 and the client 200, similarly to that according to the first embodiment. The client 200 will be described as an example by reference to FIG. 5, but similarly to the first embodiment illustrated in FIG. 1, the model generation system S2 includes plural clients 200a to 200n.

Similarly to the first embodiment, the client 200 according to the present embodiment includes an NW interface 210, a memory 220, an input interface 230, a display 240, and processing circuitry 250.

The processing circuitry 250 of the client 200 according to the present embodiment includes a start request function 241, an acquisition function 252, a training control function 253, a transmission function 254, and a report generation function 255. The report generation function 255 is an example of a report generation unit.

The start request function 251, the acquisition function 252, and the training control function 253 have functions similar to those of the first embodiment.

The report generation function 255 generates a report related to additional training that has been executed. The report will hereinafter be referred to as an additional training report. More specifically, the report generation function 255 generates an additional training report related to at least one of: training data 500 used in additional training; and a local model 350 generated by the additional training. The additional training report is an example of information related to a training log of a trainer 400 by the training control function 253.

The additional training report includes at least one of: a weight change in a model upon the additional training, statistical information on the training data 500 used in the additional training, potential feature values from the training data 500 used in the additional training, or a precision difference between the global model 300 before the training and the local model 350 after the training. The weight change in the model upon the additional training is a change in parameters between the global model 300 before the training and the local model 350 after the training. The statistical information on the training data 500 may be, for example, information representing a data distribution of the training data 500.

The additional training report does not include an entity of the training data 500. The additional training report is transmitted to the outside of the client 200. However, since the additional training report does not include the training data 500, any personal information included in the training data 500 is able to be protected.

In addition to the function according to the first embodiment, the transmission function 254 has a function of transmitting the additional training report generated by the report generation function 255 to the central server 100. The additional training report to be transmitted is correlated with the local model 350 that has been subjected to the target additional training.

Similarly to the first embodiment, the central server 100 according to the present embodiment includes an NW interface 110, a memory 120, an input interface 130, a display 140, and processing circuitry 150.

The processing circuitry 150 of the central server 100 according to the present embodiment includes a control information generation function 151, a transmission function 152, an integration function 154, an output function 155, and a determination function 156. The determination function 156 is an example of a determination unit.

The transmission function 152 has a function similar to that of the first embodiment.

In addition to the function according to the first embodiment, the acquisition function 153 according to the present embodiment has a function of acquiring the additional training report from the client 200.

The determination function 156 determines, based on the training log at the client 200, whether the local model 350 provided from this client 200 is to be used in generation of a new global model 300. More specifically, based on the additional training report acquired from the client 200, the determination function 156 determines whether the local model 350 provided from this client 200 is to be used in generation of a new global model 300. A local model 350 that has been determined, by the determination function 156, to be used in the generation of a new global model 300 will be referred to as a local model 350 to be integrated. Moreover, a local model 350 that has been determined, by the determination function 156, to be not used in the generation of a new global model 300 will be referred to as a local model 350 to be not integrated.

By using drift detection, which is a technique for evaluating machine learning, for the additional training report, for example, the determination function 156 may determine whether or not additional training having a possibility of contaminating the global model 300 has been executed. Drift detection is a technique for identifying a client 200 that has used inappropriate training data 500 by detecting a trend difference between training data 500 used in additional training at a client 200 of the plural clients 200a to 200n and training data 500 used in additional training at another client 200 of the plural clients 200a to 200n.

In a more specific example, it is supposed that in a case where the additional training report includes information representing a data distribution of the training data 500, the data distribution at a client 200 to be subjected to detection is a first data distribution, the client 200 being one of the clients 200a to 200n. Moreover, it is supposed that a data distribution resulting from integration of data distributions at the other ones of the clients 200a to 200n other than a client 200 to be subjected to detection is a second data distribution. Based on a result of a comparison between the first data distribution and the second data distribution, the determination function 156 determines whether a local model 350 provided from the client 200, from which the additional training report has been acquired, is to be used in generation of a new global model 300. For example, the determination function 156 calculates a “pseudo cost” that is a difference between the first data distribution and the second data distribution. The difference between the first data distribution and the second data distribution is also referred to as a data distribution distance.

A “pseudo cost” refers to the opposite of an “expected cost” and indicates lowness of reliability of the client 200 to be subjected to detection. Thus, the larger the difference between the first data distribution and the second data distribution, the higher the possibility determined by the determination function 156 that the training data 500 at the client 200 to be subjected to detection include data for a malicious attack or data including an error. In a case where the “pseudo cost” is higher than a threshold, the determination function 156 determines that the local model 350 acquired from the client 200 to be subjected to detection is not a target to be integrated. The “pseudo cost” is also referred to as a “pseudo score”, and the “expected cost” is also referred to as an “expected score”. The above described methods for evaluation and determination related to the additional training report are just examples, and any publicly known methods may be adopted instead.

In addition to the function according to the first embodiment, the integration function 154 according to the present embodiment has a function of integrating any local model 350 determined to be a target to be integrated by the determination function 156, into the global model 300. The integration function 154 does not integrate any local model 350 determined not to be a target to be integrated by the determination function 156, into the global model 300.

Integration processing for the local model 350, which has been determined by the determination function 156 not to be a target to be integrated, is suspended and this local model 350 is stored in the memory 120. Thereafter, in a case where a user of the central server 100 has checked a result of the determination related to this local model 350 and has performed operation to subject the local model 350 to integration, the integration function 154 may integrate the local model 350 into the global model 300.

Moreover, in addition to the function according to the first embodiment, the control information generation function 151 according to the present embodiment may have a function of changing the control information for subsequent additional training at the client 200 in accordance with a result of the determination based on the additional training report. Information recorded in the additional training report is an example of a training log of the client 200.

The control information generation function 151 may decrease the upper limit number of executions or the executable time period for the client 200 that has provided the local model 350 determined not to be a target to be integrated by the determination function 156.

Moreover, in addition to the function according to the first embodiment, the output function 155 according to the present embodiment may have a function of outputting a result of the determination by the determination function 156. For example, in a case where there is a local model 350 determined not to be a target to be integrated by the determination function 156, the output function 155 may cause the display 140 to display information enabling the client 200 to be identified, the client 200 being a client from which the local model 350 has been acquired. A user of the central server 100 is thereby able to recognize any client 200 where additional training having a possibility of contaminating the global model 300 has been executed. Moreover, the output function 155 may cause the display 140 to display the additional training report acquired from the client 200 that has provided the local model 350 determined not to be a target to be integrated, and a result of the determination by the determination function 156, the determination being related to the additional training report. The result of the determination by the determination function 156, the determination being related to the additional training report, may include, for example, information serving as a basis for the determination of the “pseudo cost” described above.

A procedure of processing for additional training executed in the model generation system S2 according to the present embodiment configured as described above will be described next.

FIG. 6 is a sequence diagram illustrating an example of a procedure of the processing for the additional training according to the second embodiment. Processing from a start request at S11 illustrated in FIG. 6 to additional training under a constraint defined by control information at S13 illustrated in FIG. 6 is similar to the processing at S1 to S3 according to the first embodiment described by reference to FIG. 4.

The report generation function 255 of the client 200 then generates an additional training report related to the additional training executed at S13 (S14).

Subsequently, the transmission function 254 of the client 200 transmits the additional training report generated at S14 to the central server 100, the additional training report having been correlated with the local model 350 generated by the additional training at S13 (S15).

The acquisition function 153 of the central server 100 then acquires the local model 350 transmitted from the client 200. Based on the additional training report acquired from the client 200, the determination function 156 of the central server 100 then determines whether or not integration of the local model 350 provided from the client 200 into the global model 300 is to be enabled (S16).

In a case where the determination function 156 has determined the local model 350 to be a target to be integrated, the integration function 154 of the central server 100 then executes integration processing of integrating the local model 350 acquired at S15 into the global model 300 (S17). In a case where the determination function 156 has determined the local model 350 not to be a target to be integrated, the integration function 154 does not integrate the local model 350 into the global model 300. Moreover, in the case where the determination function 156 has determined the local model 350 not to be a target to be integrated, the output function 155 of the central server 100 may cause the display 140 to display the following information: information enabling the client 200 to be identified, the client 200 being a client, from which the local model 350 has been acquired; the additional training report; and a result of the determination related to the additional training report.

The control information generation function 151 of the central server 100 updates the control information based on a training log of the client 200 and a result of the determination processing at S16 (S18). The updated control information is transmitted from the central server 100 to the client 200 in a subsequent round. The processing in this sequence diagram is then ended.

As described above, in the model generation system S2 according to the present embodiment, the central server 100 determines, based on the training log at the client 200, whether the local model 350 provided from the client 200 is to be used in generation of a new global model 300. Therefore, in addition to effects of the first embodiment, the model generation system S2 according to the present embodiment has an effect of reducing the influence of the local model 350 on the global model 300 even further, the local model 350 being a local model 350 that has received additional training using inappropriate training data 500.

In the model generation system S2 according to the present embodiment, the client 200 generates the additional training report related to the additional training executed and provides the central server 100 with the additional training report. Based on the additional training report, the central server 100 determines whether the local model 350 provided from the client 200 is to be used in generation of a new global model 300. Therefore, the model generation system S2 according to the present embodiment enables evaluation of the content of the additional training and determination on whether or not integration of the local model 350 is to be enabled, without transmission of the training data 500 themselves outside the client 200.

Moreover, in the model generation system S2 according to the present embodiment, the central server 100 changes the control information for subsequent additional training at the client 200 in accordance with a result of the determination based on the additional training report. Therefore, the model generation system S2 according to the present embodiment enables reduction of chances of inappropriate additional training in a subsequent round.

Moreover, in the model generation system S2 according to the present embodiment, the additional training report includes information representing a data distribution of the training data 500. Based on a result of a comparison between the first data distribution at a client 200, from which the additional training report has been acquired, and the second data distribution resulting from integration of the data distributions of the other ones of the clients 200a to 200n excluding the client 200, the central server 100 determines whether the local model 350 provided from the client 200 is to be used in generation of a new global model 300. Therefore, the model generation system S2 according to the present embodiment enables specification of the client 200 that has used inappropriate training data 500.

### Third Embodiment

In the first and second embodiments described above, the client 200 includes the trainer 400 beforehand, but a trainer 400 assigned with control information may be provided from the central server 100 to the client 200.

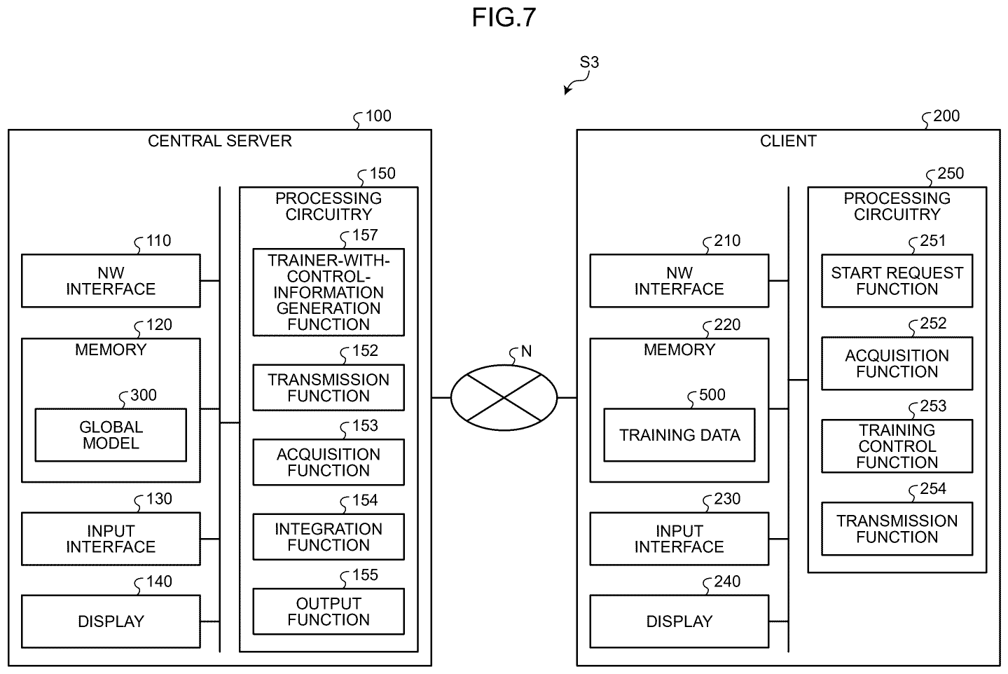

FIG. 7 is a block diagram illustrating an example of a configuration of a central server 100 and a client 200, according to a third embodiment.

A model generation system S3 according to the present embodiment includes the central server 100 and the client 200, similarly to those according to the first and second embodiments. The client 200 will be described as an example by reference to FIG. 7, but similarly to the first embodiment illustrated in FIG. 1, the model generation system S3 includes plural clients 200a to 200n.

Similarly to the first and second embodiments, the client 200 according to the present embodiment includes an NW interface 210, a memory 220, an input interface 230, a display 240, and processing circuitry 250.

The processing circuitry 250 at the client 200 according to the present embodiment includes a start request function 241, an acquisition function 252, a training control function 253, and a transmission function 254.

The start request function 251 and the transmission function 254 have functions similar to those of the first embodiment.

In addition to the function according to the first embodiment, the acquisition function 252 according to the present embodiment has a function of acquiring a trainer 400 that has been assigned with control information, from the central server 100. The trainer 400 that has been assigned with the control information will hereinafter be referred to as the trainer 400 with control information.

The training control function 253 executes additional training by using the trainer 400 with control information transmitted from the central server 100.

Moreover, similarly to the first embodiment, the central server 100 according to the present embodiment includes an NW interface 110, a memory 120, an input interface 130, a display 140, and processing circuitry 150.

The processing circuitry 150 of the central server 100 according to the present embodiment includes a trainer-with-control-information generation function 157, a transmission function 152, an integration function 154, and an output function 155. The trainer-with-control-information generation function 157 is an example of a control information generation unit and a trainer-with-control-information generation unit.

The integration function 154 and the output function 155 have functions similar to those of the first embodiment.

The trainer-with-control-information generation function 157 generates a trainer with control information 400. More specifically, the trainer-with-control-information generation function 157 has a function of generating control information similarly to the control information generation function 151 according to the first embodiment and a function of assigning the control information generated to a trainer 400.

The trainer with control information 400 is an application program incorporated with control information defining a constraint limiting, for example, an upper limit number of executions or an executable time period.

The executable time period may be assigned as a license with an expiration date to the trainer with control information 400. In this case, after the executable time period for the trainer with control information 400 provided to the client 200 has elapsed, the trainer with control information 400 may be automatically deleted from the client 200. Alternatively, after the executable time period for the trainer with control information 400 provided to the client 200 has elapsed, the trainer with control information 400 may be brought into a state of being unable to be executed on the client 200.

The control information assigned to the trainer with control information 400 may constrain the trainer with control information 400 from being able to be executed at clients 200 other than the client 200, to which the trainer 400 with control information 400 has been transmitted.

In addition to the function according to the first embodiment, the transmission function 152 according to the present embodiment has a function of transmitting the trainer with control information 400 generated by the trainer-with-control-information generation function 157, to the client 200.

A procedure of processing for additional training executed in the model generation system S3 according to the present embodiment configured as described above will be described next.

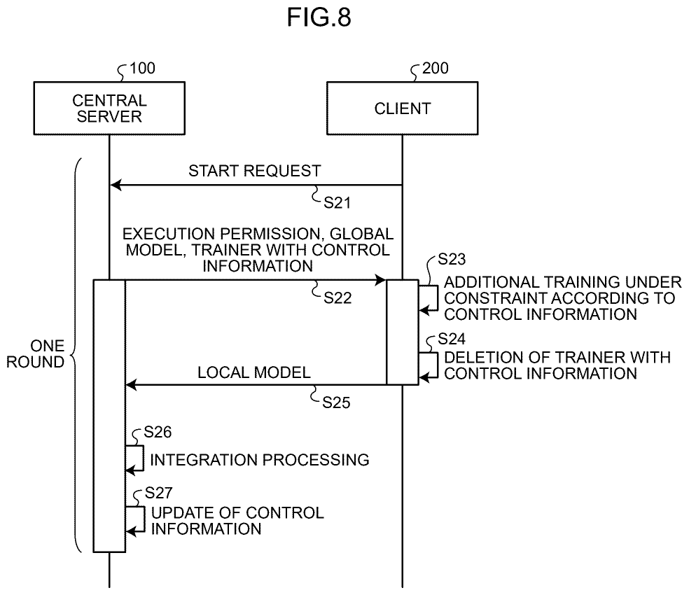

FIG. 8 is a sequence diagram illustrating an example of a procedure of the processing for the additional training according to the third embodiment. As a premise of this processing illustrated in FIG. 8, it is supposed that the trainer-with-control-information generation function 157 of the central server 100 has already generated the trainer with control information 400.

Processing for a start request at S21 illustrated in FIG. 8 is similar to the processing at S1 according to the first embodiment described by reference to FIG. 4.

Upon receipt of a start request from the client 200, the transmission function 152 of the central server 100 transmits permission for execution of additional training, a global model 300, and the trainer with control information 400, to the client 200 (S22).

The acquisition function 252 of the client 200 acquires the permission for execution of additional training, the global model 300, and the trainer with control information 400, which have been transmitted from the central server 100. The trainer with control information 400 is stored in the memory 220 and installed on the client 200. The training control function 253 of the client 200 activates the trainer with control information 400 and provides training data 500 to the trainer with control information 400 to cause the trainer with control information 400 to execute training based on the control information (S23).

In a case where the control information on the trainer with control information 400 includes an executable time period for the trainer 400 as an expiration date of the trainer 400 and defines that the trainer 400 is to be deleted after the expiration date, the trainer with control information 400 is deleted from the client 200 after the executable time period elapses (S24). The processing at S24 differs with contents of the control information on the trainer with control information 400. In one example, if the control information prohibits the trainer 400 from being executed after the executable time period, the trainer with control information 400 will not be able to be activated after the executable time period.

Transmission of a local model 350 at S25 to integration processing at S26 are similar to the processing at S4 and S5 according to the first embodiment described by reference to FIG. 4.

The trainer-with-control-information generation function 157 then updates the control information in accordance with a training log of the training at the client 200 and generates a new trainer with control information 400 (S27). The new trainer with control information 400 is transmitted from the central server 100 to the client 200 in a subsequent round. The processing at S27 is not indispensable. The processing in this sequence diagram is then ended.

As described above, in the model generation system S3 according to the present embodiment, the client 200 acquires the trainer with control information 400 from the central server 100. In addition to the effects according to the first embodiment, the model generation system S3 according to the present embodiment has an effect of enabling abuse of the trainer 400 to be reduced, because the control information has been incorporated into the trainer 400 itself.

### Fourth Embodiment

In the first to third embodiments described above, the central server 100 has been described as an example of the computer that is capable of providing the clients 200 with the global model 300 to be used in federated learning. By contrast, in this fourth embodiment, an X-ray computer tomography (CT) apparatus provides clients 200 with a global model 300 to be used in federated learning.

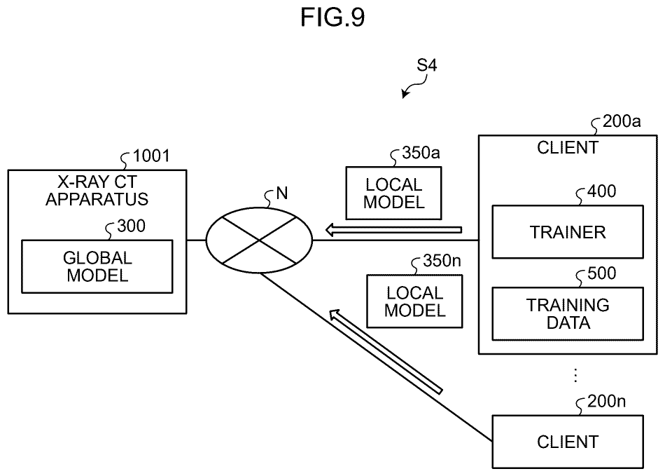

FIG. 9 is a diagram illustrating an example of a configuration of a model generation system S4 according to a fourth embodiment. As illustrated in FIG. 9, the model generation system S4 according to the present embodiment includes an X-ray CT apparatus 1001 and plural clients 200a to 200n (hereinafter, the clients 200). The X-ray CT apparatus 1001 and the clients 200 are connected to be able to communicate with each other via a network N, such as the Internet, for example.

The X-ray CT apparatus 1001 according to the present embodiment has the global model 300 to be used in federated learning. The X-ray CT apparatus 1001 provides the clients 200 with the global model 300 to be used in federated learning. The X-ray CT apparatus 1001 is an example of an information processing apparatus according to the present embodiment. Moreover, the X-ray CT apparatus 1001 may be called a first information processing apparatus, and a client 200 may be called a second information processing apparatus. Either one of the X-ray CT apparatus 1001 or a client 200 may be called an information processing apparatus, and the other one may be called another information processing apparatus.

A client 200 according to the present embodiment is configured similarly to that according to the first embodiment. For example, the client 200 applies the global model 300 acquired from the X-ray CT apparatus 1001 to a trainer 400. The client 200 generates a local model 350 by providing the trainer 400 with training data 500. The client 200 is capable of providing the X-ray CT apparatus 1001 with the generated local model 350. Moreover, a constraint related to training is placed by the X-ray CT apparatus 1001 on each of the clients 200a to 200n. The client 200 according to the present embodiments may include functions similar to those according to the second embodiment or third embodiment.

Similarly to the first embodiment, the constraint is an upper limit number of executions or an executable time period for a trainer 400 at each of the clients 200a to 200n. The X-ray CT apparatus 1001 transmits control information for controlling the trainers 400 based on the constraints, to the clients 200a to 200n. Therefore, execution of the trainers 400 at the clients 200a to 200n is controlled by the constraints assigned to the trainers 400.

The global model 300 according to the present embodiment may be an AI model that has been trained with training data including X-ray CT image data, for example. Moreover, training data for the local model 350 according to the present embodiment may include X-ray CT image data stored at each client 200. For example, training data for the global model 300 and the local model 350 may be data obtained by correlating X-ray CT image data with information representing a range of an abnormal portion, such as a lesion, in the X-ray CT image data. These pieces of training data are just an example, and the training data for the global model 300 and the local model 350 are not to be limited to this example. The X-ray CT image data included in the training data may be those captured by the X-ray CT apparatus 1001 having the global model 300 or may be those captured by another X-ray CT apparatus.

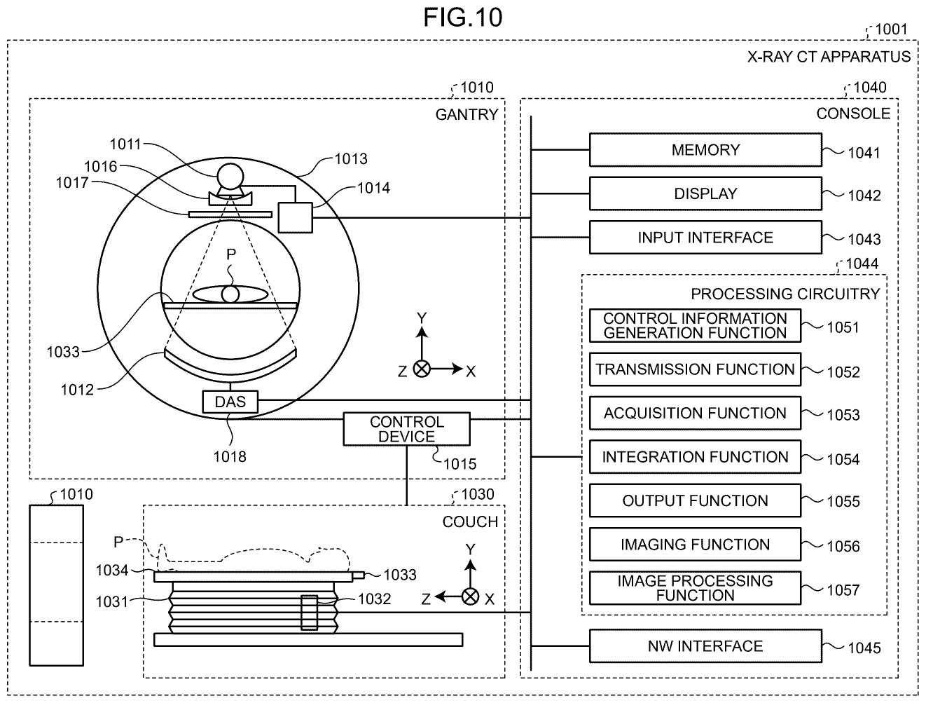

FIG. 10 is a diagram illustrating an example of a configuration of the X-ray CT apparatus 1001 according to the fourth embodiment. In the X-ray CT apparatus 1001, X-rays are emitted to a subject (or a patient) P from an X-ray tube 1011 and the X rays emitted are detected by an X-ray detector 1012. Based on output from the X-ray detector 1012, the X-ray CT apparatus 1001 generates a CT image related to the subject P. Various types of X-ray CT apparatuses, such as those of the third generation CT, the fourth generation CT, and the fifth generation CT, are available, but any type of X-ray CT apparatus is applicable to the present embodiment and may be used as the X-ray CT apparatus 1001. The third generation CT is a rotate/rotate-type, in which an X-ray tube and a detector rotate in integration with each other around a subject. The fourth generation CT is a stationary/rotate-type, in which multiple X-ray detection elements arrayed in a ring shape are fixed and only an X-ray tube rotates around a subject. The fifth generation system includes a focus coil that focuses an electron beam generated by an electron gun, a deflection coil that causes electromagnetic deflection of the focused electron beam, and a target ring that surrounds halfway around a subject P and generates X-rays through collision of the deflected electron beam against the target ring.

As illustrated in FIG. 10, the X-ray CT apparatus 1001 has a gantry 1010, a couch 1030, and a console 1040. For illustrative purposes, FIG. 10 has plural gantries 1010 depicted therein. The gantry 1010 is a scan apparatus having a configuration for subjecting a subject P to an X-ray CT scan. The couch 1030 is a conveyance apparatus for having, placed thereon, the subject P to be subjected to the X-ray CT scan and positioning the subject P. The console 1040 is a computer that controls the gantry 1010. For example, the gantry 1010 and the couch 1030 are installed in a CT examination room and the console 1040 is installed in a control room adjacent to the CT examination room. The gantry 1010, the couch 1030, and the console 1040 are connected by wire or wirelessly to be able to communicated with one another.

The console 1040 is not necessarily installed in the control room. In one example, the console 1040 may be installed in the same room as the gantry 1010 and the couch 1030. The console 1040 may be incorporated into the gantry 1010.

In the present embodiment, in a non-tilted state, a direction along a rotation axis of a rotation frame 1013 or a longitudinal direction of a couchtop 1033 of the couch 1030 is defined as a Z-axis direction, an axial direction orthogonal to the Z-axis direction and horizontal to a floor surface as an X-axis direction, and an axial direction orthogonal to the Z-axis direction and perpendicular to the floor surface as a Y-axis direction.

As illustrated in FIG. 10, the gantry 1010 has the X-ray tube 1011, the X-ray detector 1012, the rotation frame 1013, an X-ray high voltage generator 1014, a control device 1015, a wedge 1016, a collimator 1017, and a data acquisition system (DAS) 1018.

The X-ray tube 1011 is a vacuum tube having: a cathode (filament) that generates thermions; and an anode (target) that generates X-rays upon collision of the thermions against the anode. The X-ray tube 1011 emits the X-rays to the subject P by emitting the thermions from the cathode to the anode using high voltage supplied from the X-ray high voltage generator 1014.

Hardware to generate the X-rays is not necessarily the X-ray tube 1011. For example, instead of having the X-ray tube 1011, X-rays may be generated using the fifth generation system.

The X-ray detector 1012 detects X-rays that have been emitted from the X-ray tube 1011 and passed through the subject P and outputs an electric signal corresponding to a dose of the X-rays detected, to the DAS 1018. The X-ray detector 1012 has, for example, an X-ray detection element array having plural X-ray detection elements arrayed in a channel direction along one circular arc about a focus of the X-ray tube 1011. The X-ray detector 1012 has, for example, a structure, in which the plural X-ray detection elements in the channel direction are arrayed plurally in a slice direction (column direction and/or row direction). The X-ray detector 1012 is, for example, an indirect conversion detector having a grid, a scintillator array, and an optical sensor array. The scintillator array has plural scintillators. The scintillators have scintillator crystal that outputs light of a quantity corresponding to a dose of X-rays incident thereon. The grid is arranged on a surface of the scintillator array, the surface being where the X-rays are incident on, and has an X-ray shield plate having a function of absorbing scattered X-rays. The grid may also be called a collimator (one-dimensional collimator or two-dimensional collimator). The optical sensor array has a function of converting light from the scintillator into an electric signal corresponding to a quantity of the light. Photomultipliers (PMTs) are used as optical sensors, for example. The X-ray detector 1012 may be a direct conversion detector having a semiconductor element that converts X-rays incident thereon into an electric signal. The X-ray detector 1012 is an example of a detection unit.

The rotation frame 1013 is an annular frame that supports the X-ray tube 1011 and the X-ray detector 1012 opposed to each other and rotates the X-ray tube 1011 and the X-ray detector 1012 by means of the control device 1015 described later. A field of view (FOV) is set for an opening in the rotation frame 1013. For example, the rotation frame 1013 is a casting made from aluminum. The rotation frame 1013 may further support, in addition to the X-ray tube 1011 and the X-ray detector 1012, for example: the X-ray high voltage generator 1014; the wedge 1016; the collimator 1017, and/or the DAS 1018. The rotation frame 1013 may further support any of various components not illustrated in FIG. 10.

The X-ray high voltage generator 1014 has a high voltage generator and an X-ray controller. The high voltage generator has electric circuitry including a transformer and a rectifier, and generates high voltage to be applied to the X-ray tube 1011 and filament current to be supplied to the X-ray tube 1011. The X-ray controller controls output voltage in accordance with X-rays emitted by the X-ray tube 1011. The high voltage generator may be of the transformer-type or the inverter-type. The X-ray high voltage generator 1014 may be installed on the rotation frame 1013 in the gantry 1010 or installed on a fixed frame (not illustrated in the drawings) in the gantry 1010. The fixed frame is a frame that supports the rotation frame 1013 rotatably.

The control device 1015 includes: a drive mechanism including a motor and an actuator; and processing circuitry having: a processor that controls this drive mechanism; and a memory, for example. The control device 1015 executes controls operation of the gantry 1010 and the couch 1030 by receiving input signals from an input interface 1043 or another input interface provided in the gantry 1010, for example. The control of the operation by the control device 1015 includes, for example, control to rotate the rotation frame 1013, control to tilt the gantry 1010, and control to operate the couch 1030. The control to tilt the gantry 1010 is implemented by the control device 1015 rotating the rotation frame 1013 about an axis parallel to the X-axis direction according to inclination angle (tilt angle) information input by the input interface 1043 mounted on the gantry 1010. The control device 1015 may be provided in the gantry 1010 or provided in the console 1040.

The wedge 1016 is a filter for adjusting the dose of X-rays emitted from the X-ray tube 1011. Specifically, the wedge 1016 is a filter that transmits and attenuates X-rays therethrough, the X-rays having been emitted from the X-ray tube 1011, so that the X rays emitted to the subject P from the X-ray tube 1011 have a predetermined distribution. The wedge 1016 may be a wedge filter or a bow-tie filter, and is formed by processing, for example, aluminum to achieve a predetermined target angle and/or to have a predetermined thickness.

The collimator 1017 limits a range irradiated with X-rays that have been transmitted through the wedge 1016. The collimator 1017 slidably supports plural lead plates that block X-rays and adjusts the form of slits formed by the plural lead plates. The collimator 1017 may be called an X-ray aperture.

The DAS 1018 reads an electric signal corresponding to a dose of X-rays detected by the X-ray detector 1012, from the X-ray detector 1012. The DAS 1018 collects detection data having a digital value corresponding to a dose of X-rays over a viewing time period by amplifying the electric signal read and integrating (adding up) the electric signal over the viewing time period. The detection data is called projection data. The DAS 1018 is implemented by, for example, an application specific integrated circuit (ASIC) having, installed thereon, circuit elements enabling the projection data to be generated. The projection data is transmitted to the console 1040 via a non-contact data transmission device, for example. The DAS 1018 is an example of a detection unit.

The detection data generated by the DAS 1018 are transmitted to a receiver having a photodiode and then transferred to the console 1040 by optical communication from a transmitter having a light emitting diode (LED) provided on the rotation frame 1013, the receiver having been provided in a non-rotating portion (for example, the fixed frame of the gantry 1010, illustration of the fixed frame being omitted in FIG. 10) of the gantry 1010. A method of transmitting the detection data to the non-rotating portion of the gantry 1010 from the rotation frame 1013 of a rotating portion is not limited to the optical communication described above, and any method of performing non-contact data transfer may be adopted.

In the present embodiment, the X-ray CT apparatus 1001 having the integrating X-ray detector 1012 installed therein is described as an example, but a technique according to the present embodiment may be implemented as an X-ray CT apparatus 1001 having a photon-counting X-ray detector installed therein.

The couch 1030 is an apparatus that the subject P to be scanned is placed on and that moves the subject P to be scanned. The couch 1030 has a base 1031, a couch driver 1032, the couchtop 1033, and a support frame 1034. The base 1031 is a housing that supports the support frame 1034 movably in a vertical direction. The couch driver 1032 is a drive mechanism that moves the couchtop 1033, on which the subject P has been placed, in the longitudinal direction of the couchtop 1033. The couch driver 1032 includes, for example, a motor and an actuator. The couchtop 1033 is a board where the subject P is to be placed on. The couchtop 1033 is provided on an upper surface of the support frame 1034. The couchtop 1033 is capable of protruding toward the gantry 1010 from the couch 1030 so that the whole body of the subject P is able to be scanned. The couchtop 1033 is formed of, for example, carbon fiber reinforced plastic (CFRP) high in X-ray transmissivity and having good physical properties, such as rigidity and strength. The couchtop 1033 may be hollow on the inside. The support frame 1034 supports the couchtop 1033 movably in the longitudinal direction of the couchtop 1033. The couch driver 1032 may move, in addition to the couchtop 1033, the support frame 1034 in the longitudinal direction of the couchtop 1033.

The console 1040 has a memory 1041, a display 1042, the input interface 1043, processing circuitry 1044, and an NW interface 1045. Data communication between the memory 1041, the display 1042, the input interface 1043, the NW interface 1045, and the processing circuitry 1044 is performed via a bus. The console 1040 is described as being separate from the gantry 1010, but the console 1040 or part of components of the console 1040 may be included in the gantry 1010.

The memory 1041 is implemented by, for example, a semiconductor memory element, such as a ROM, a RAM, a flash memory, or the like, a hard disk, an optical disk, etc. The memory 1041 stores various kinds of data to be stored. The memory 1041 stores projection data and reconstructed image data. The memory 1041 stores various programs, for example. A storage area of the memory 1041 may be in the X-ray CT apparatus 1001 or in an external storage device connected via a network. Moreover, the memory 1041 stores the trainer 400 and the local model 350, for example.

The display 1042 is, for example, a liquid crystal display or a CRT display that displays various kinds of information.

The display 1042 may be installed at any place in the control room. The display 1042 may be installed on the gantry 1010. The display 1042 may be of the desktop type, or may be formed of a tablet terminal capable of wirelessly communicating with the console 1040. Moreover, one projector, or two or more projectors may be used as the display 1042.

The input interface 1043 receives various kinds of input operation from an operator, converts the input operation received, into electric signals, and outputs the electric signals to the processing circuitry 1044.

Any of a mouse, a keyboard, a trackball, switches, buttons, a joystick, a touchpad, and a touch panel display, for example, may be used as the input interface 1043. In the present embodiment, the input interface 1043 does not necessarily have any of these physical operating parts. Examples of the input interface 1043 include electric signal processing circuitry that receives an electric signal corresponding to input operation from an external input device provided separately from the apparatus and outputs this electric signal to the processing circuitry 1044. The input interface 1043 may be provided on the gantry 1010. The input interface 1043 may be formed of, for example, a tablet terminal that is capable of wirelessly communicating with the main body of the console 1040. The input interface 1043 is an example of an input unit.

The NW interface 1045 has been connected to the processing circuitry 1044 and controls transmission and communication of various data performed between the X-ray CT apparatus 1001 and the clients 200. The NW interface 1045 is implemented by a network card, a network adapter, or an NIC, for example.

The processing circuitry 1044 controls the overall operation of the X-ray CT apparatus 1001. The processing circuitry 1044 has hardware resources including a processor and memories, such as a ROM and a RAM. The processing circuitry 1044 executes various kinds of processing for the system by the processor that executes programs loaded into a memory. The processing circuitry 1044 includes, for example, a control information generation function 1051, a transmission function 1052, an acquisition function 1053, an integration function 1054, and an output function 1055. The control information generation function 1051, the transmission function 1052, the acquisition function 1053, the integration function 1054, and the output function 1055 include functions similar to the control information generation function 151, the transmission function 152, the acquisition function 153, the integration function 154, and the output function 155 in the processing circuitry 150 of the central server 100 according to the first embodiment described by reference to FIG. 2. The processing circuitry 1044 may include the determination function 156 according to the second embodiment described by reference to FIG. 5. Moreover, the processing circuitry 1044 may include the trainer-with-control-information generation function 157 according to the third embodiment described by reference to FIG. 7.

The processing circuitry 1044 further includes an imaging function 1056 and an image processing function 1057. The imaging function 1056 and the image processing function 1057 are functions for capturing X-ray CT images.

The imaging function 1056 acquires data of X-rays detected by the X-ray detector 1012 by controlling a CT scan executed at the gantry 1010. The imaging function 1056 acquires X-ray CT image data resulting from imaging of the subject P by performing reconstruction processing of the detection data of the X-rays. Examples of the reconstruction processing include filtered back projection, iterative reconstruction, and machine learning. Based on input operation received from an operator via the input interface 1043, the image processing function 1057 converts X-ray CT image data to tomographic image data or three-dimensional image data of any cross section by a publicly known method.

The control information generation function 1051 according to the present embodiment generates control information defining constraints to be placed on the clients 200.

The transmission function 1052 according to the present embodiment transmits permission for execution of additional training, the global model 300, and the control information, to a client 200, via the NW interface 110 and the network N.

The acquisition function 1053 according to the present embodiment acquires a start request for the additional training from the client 200. Moreover, the acquisition function 1053 acquires a local model 350 from the client 200.

The integration function 1054 according to the present embodiment integrates the local model 350 acquired from the client 200 by the acquisition function 1053, into the global model 300.

The output function 1055 according to the present embodiment controls the display 1042 to cause the display 1042 to display various kinds of information. For example, the output function 1055 may cause the display 1042 to display information representing that the integration of the local model 350 into the global model 300 has been completed. The output function 1055 according to the present embodiment may input X-ray CT image data captured and generated by the imaging function 1056 and the image processing function 1057, to the global model 300, and cause the display 1042 to display information related to the X-ray CT image data output from the global model 300. For example, in a case where training data is data having X-ray CT image data and information correlated with each other, the information representing a range of an abnormal portion, such as a lesion, in the X-ray CT image data, as described above, the global model 300 outputs information representing a range of an abnormal portion, such as a lesion, in X-ray CT image data upon receipt of input of the X-ray CT image data. In this case, the output function 1055 may cause the display 1042 to display the information representing the range of the abnormal portion, such as a lesion, in the X-ray CT image data, the information having been output from the global model 300.

As described above, in the present embodiment, the X-ray CT apparatus 1001 has the global model 300, and additional training in federated learning is able to be implemented by integration of the local model 350 trained at the client 200 into the global model 300 under control of the X-ray CT apparatus 1001. Therefore, the X-ray CT apparatus 1001 according to the present embodiment has, in addition to effects similar to those according to the first embodiment, an effect of enabling, through additional training, improvement in precision of the global model 300 that the X-ray CT apparatus 1001 has.

In the present embodiment, the X-ray CT apparatus 1001 is an example of a computer capable of providing the client 200 with the global model 300 to be used in federated learning, but any other medical diagnostic imaging apparatus may be an example of the computer. For example, any of various medical diagnostic imaging apparatuses may have the functions of the processing circuitry 1044 illustrated in FIG. 10, the various medical diagnostic imaging apparatuses including an X-ray diagnostic apparatus, a magnetic resonance imaging (MRI) apparatus, an ultrasound diagnostic apparatus, a single photon emission computed tomography (SPECT) apparatus, a positron emission computed tomography (PET) apparatus, a SPECT-CT apparatus having a SPECT apparatus and an X-ray CT apparatus integrated together, and a PET-CT apparatus having a PET apparatus and an X-ray CT apparatus integrated together.

### Fifth Embodiment

In the above described fourth embodiment, the X-ray CT apparatus 1001 has the global model 300. By contrast, an X-ray CT apparatus 1001 in a fifth embodiment uses a global model 300 that a central server 100 has.

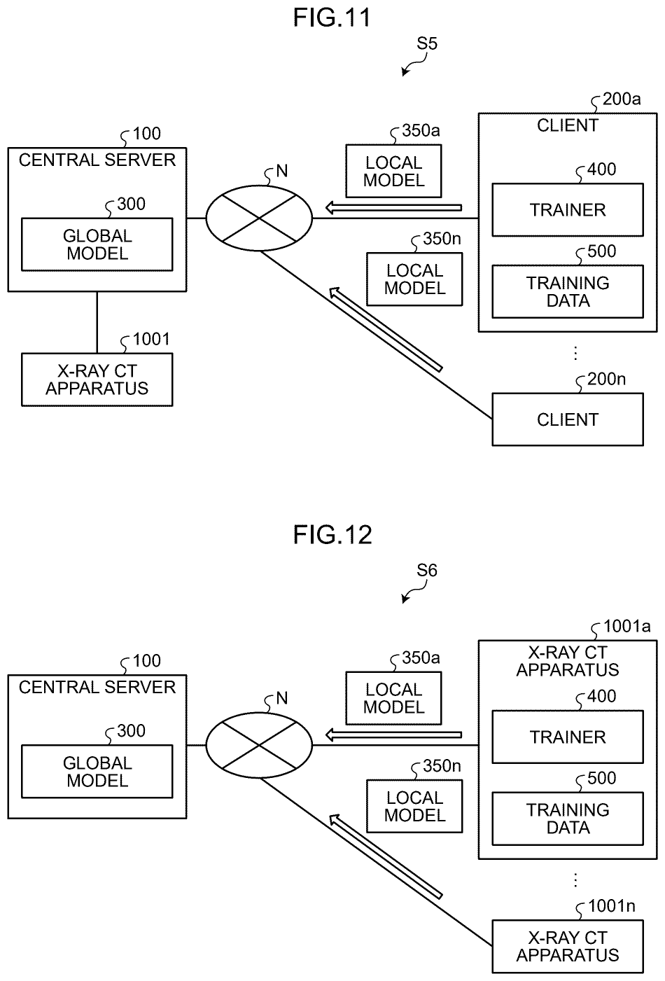

FIG. 11 is a diagram illustrating an example of a configuration of a model generation system S5 according to the fifth embodiment.

As illustrated in FIG. 11, the model generation system S5 according to the present embodiment includes the central server 100, the X-ray CT apparatus 1001, and plural clients 200a to 200n (hereinafter, clients 200). The central server 100 and the clients 200 are connected to be able to communicate with each other via a network N, such as the Internet, for example, similarly to the first embodiment.

In the model generation system S5 according to the present embodiment, the X-ray CT apparatus 1001 is communicably connected to the central server 100. In FIG. 11, the X-ray CT apparatus 1001 is separately connected to the central server 100, but the X-ray CT apparatus 1001 and the central server 100 may be connected via the network N, such as the Internet.

The central server 100 according to the present embodiment has a configuration similar to that according to the first embodiment. The central server 100 according to the present embodiment may have functions similar to those according to the second embodiment or third embodiment.

The clients 200 according to the present embodiment each have a configuration similar to that according to the first embodiment. The clients 200 according to the present embodiment may each have a configuration similar to that according to the second embodiment or third embodiment.

The X-ray CT apparatus 1001 according to the present embodiment has a hardware configuration similar to the configuration according to the fourth embodiment described by reference to FIG. 10. Moreover, the processing circuitry 1044 of the X-ray CT apparatus 1001 according to the present embodiment may be without the control information generation function 1051, the transmission function 1052, the acquisition function 1053, and the integration function 1054, of the plural functions illustrated in FIG. 10. For example, the processing circuitry 1044 of the X-ray CT apparatus 1001 according to the present embodiment may include an output function 1055, an imaging function 1056, and an image processing function 1057.

The output function 1055 according to the present embodiment may input X-ray CT image data captured and generated by the imaging function 1056 and the image processing function 1057 to a global model 300 in the central server 100. In this case, the output function 1055 may cause a display 1042 to display information related to the X-ray CT image data output from the global model 300.

### Sixth Embodiment

In the above described fourth embodiment, the X-ray CT apparatus 1001 has the role of the central server 100 according to the first embodiment. By contrast, in a sixth embodiment, an X-ray CT apparatus 1001 has a role of a client 200 according to the first embodiment.

FIG. 12 is a diagram illustrating an example of a configuration of a model generation system S6 according to the sixth embodiment.

As illustrated in FIG. 12, the model generation system S6 according to the present embodiment includes a central server 100 and plural X-ray CT apparatuses 1001a to 1001n. In a case where the individual X-ray CT apparatuses 1001a to 1001n are not distinguished from one another, they will hereinafter be simply referred to as X-ray CT apparatuses 1001. The central server 100 and the X-ray CT apparatuses 1001 are connected to be able to communicate with each other via a network N, such as the Internet, for example.

The central server 100 according to the present embodiment has a configuration similar to that according to the first embodiment. Alternatively, the central server 100 according to the present embodiment may have a configuration similar to that according to the second embodiment or third embodiment.

The X-ray CT apparatus 1001 according to the present embodiment has a hardware configuration similar to the configuration according to the fourth embodiment described by reference to FIG. 10.

The X-ray CT apparatuses 1001 according to the present embodiment each have functions similar to those of the clients 200 according to the first embodiment, in addition to an imaging function 1056 and an image processing function 1057 similar to those according to the fourth embodiment. For example, processing circuitry 1044 of an X-ray CT apparatus 1001 according to the present embodiment may include functions similar to the start request function 251, the acquisition function 252, the training control function 253, and the transmission function 254, of the processing circuitry 250 described by reference to FIG. 3.

Moreover, the X-ray CT apparatuses 1001 according to the present embodiment may each have functions similar to those of the clients 200 according to the second embodiment or third embodiment.

In the model generation system S6 according to the present embodiment, each of the plural X-ray CT apparatuses 1001a to 1001n generates a local model 350 by providing a trainer 400 with training data 500. Therefore, training using X-ray CT image data is able to be executed at the X-ray CT apparatuses 1001 without provision of clients 200 dedicated to training processing.

Moreover, the model generation system S6 may include, instead of the central server 100, an X-ray CT apparatus 1001 having a global model 300. That is, the model generation system S6 may include one X-ray CT apparatus 1001 having a global model 300 and the plural X-ray CT apparatuses 1001a to 1001n each including a trainer 400.

### First Modified Example

In each of the embodiments described above, each client 200 corresponds to one patient, but a client or clients 200 may correspond to an enterprise that provides PHRs. Each set of training data 500 may correspond to medical data about one patient. In this case, a constraint to be placed on a client 200 may be an upper limit number of sets of training data 500 to be used in additional training of one round, for example.

The plural clients 200a to 200n may be not independent as hardware. For example, plural clients 200a to 200n for respective patients may be present in one server managed by an enterprise for PHRs.

### Second Modified Example

In each of the foregoing embodiments, data of PHRs is described as an example of the training data 500, but the training data 500 are not limited to this example. For example, the training data 500 may be medical data, such as electronic medical records stored in a medical institution, such as a hospital. In this case, the clients 200 may be, for example, medical institutions. Moreover, the model generation systems S1 to S3 may be applied to federated learning in any field other than the medical field.

### Third Modified Example

In each of the foregoing embodiments, the constraint is the upper limit number of executions or the executable time period for the trainer 400 but the constraint is not limited to this example. For example, the control information may include a constraint that limits a memory size to be used in additional training by the trainer 400. The number of sets of data that are able to be used in additional training per unit time may vary with differences between environments of the clients 200. Therefore, the control information prescribing the executable time period and the memory size to be used in the additional training enables the central server 100 to control the amounts of the additional training at the clients 200 even more precisely.

### Fourth Modified Example

In each of the foregoing embodiments, the case where storage of training data 500 required for additional training in a subsequent round in the memory 120 serves as a condition for the client 200 to issue a start request serving as a trigger for start of additional training has been described. However, the condition for issuing the start request is not limited to this example. For example, the start request may be transmitted at any timing in response to operation by a user of the client 200.

Moreover, the central server 100 may receive the start request from the client 200 only during prescribed hours of day.

### Fifth Modified Example

In each of the foregoing embodiments, the configuration that automatically executes additional training in the case where the training control function 253 at the client 200 has received permission for execution of the additional training from the central server 100 has been described, but the means for permitting execution of additional training is not limited to this configuration.

For example, in a case where additional training at a client 200 is executed under operation by a user, permission for execution of the additional training may be a message to the user of the client 200. The client 200 that has received the message may let the user know that the additional training is able to be executed by outputting a notification to the display 240, for example.

In another method, the transmission function 152 of the central server 100 may transmit a one-time password as the permission for the execution of the additional training, to the client 200. The one-time password grants a license to the trainer 400 of the client 200.

### Sixth Modified Example

In the second foregoing embodiments, the determination using the additional training report has been described as an example, the determination being on whether or not integration of the local model 350 is to be enabled, but the method for this determination is not limited to this example.

As another determination method, the determination function 156 of the central server 100 may input test input data to the local model 350, and make an evaluation of performance of the local model 350 based on an output result from the local model 350. In this case, the determination function 156 determines, based on a result of the evaluation, whether or not integration of the local model 350 is to be enabled.

Specifically, the determination function 156 calculates an expected score of the local model 350 based on the output result from the local model 350, the output result corresponding to the test input data. For example, the determination function 156 may compare true data corresponding to the test input data and the output result from the local model 350 and, the closer the output result to the true data, the higher the performance of the local model 350 determined by the determination function 156. The higher the performance of the local model 350, the higher the expected score calculated. In a case where the expected score of the local model 350 is equal to or higher than a threshold, the determination function 156 may determine the local model 350 as a target to be integrated and in a case where the expected score is lower than the threshold, the determination function 156 may determine the local model 350 to be not a target to be integrated. The test input data and the true data may be stored in the memory 120 of the central server 100 beforehand. If this configuration is adopted, the client 200 may be without the function to generate the additional training report. Alternatively, the determination function 156 of the central server 100 may use both a determination using the additional training report and an evaluation of performance using the test data.

The expected score of the local model 350 may be used in update of the control information in the first, second, or third embodiment. The control information generation function 151 may extend a trainable time period in a subsequent round or increase the upper limit number of executions for the trainer 400, for a client 200 that provides a local model 350 and has an expected score equal to or higher than a prescribed threshold. The threshold used in the determination on whether or not integration of the local model 350 is to be enabled and the threshold for determining the constraint in the subsequent round may be different from each other.

### Seventh Modified Example

The description of the third embodiment is based on the configuration according to the first embodiment, but the configuration according to the second embodiment and the configuration according to the third embodiment may be combined with each other. For example, a configuration, in which a central server 100 determines whether or not integration of a local model 350 is to be enabled, like in the second embodiment, and a configuration, in which a trainer with control information 400 is provided from a central server 100 to a client 200, like in the third embodiment, may be combined with each other.

### Eighth Modified Example

The central servers 100 in the model generation systems S1 to S3, S5, and S6 and the X-ray CT apparatus 1001 in the model generation system S4 may each further include an inference function that executes inference processing with the global model 300. For example, the inference function acquires input data for inference and inputs the input data to the global model 300, and acquires output data output from the global model 300. Alternatively, the model generation systems S1 to S6 may each provide the generated global model 300, to another system or information processing apparatus.

In this specification, the various kinds of data handled are typically digital data.

According to at least one of the embodiments described above, quality of additional training is able to be maintained in federated learning.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray CT apparatus, comprising:
an X-ray tube configured to emit X-rays to a subject;
an X-ray detector configured to detect X-rays emitted from the X-ray tube;
at least one memory configured to store a global model to be used in federated learning; and
at least one piece of processing circuitry connected to the memory and configured to
generate CT image data by executing reconstruction processing on detection data of the X-rays detected by the X-ray detector,
transmit, to a client, the global model and control information controlling execution of a trainer at the client,
acquire, from the client, a local model generated by training of the global model with training data by the trainer under control of the control information, and
update the control information in accordance with a training log of the client.

2. A model generation system, comprising:
a client capable of executing a trainer; and
a central server capable of providing the client with a global model to be used in federated learning, wherein
the client is configured to
apply, to the trainer, the global model acquired from the central server,
generate a local model by inputting training data to the trainer, and
provide the central server with the local model,
execution of the trainer at the client is controlled by control information assigned to the trainer, and
the central server is configured to execute control to enable the control information to be changed with a training log of the client.

3. The model generation system according to claim 2, wherein the control information is information defining, for the trainer, an upper limit number of executions or an executable time period.

4. The model generation system according to claim 2, wherein the client is configured to acquire the trainer assigned with the control information from the central server.

5. The model generation system according to claim 2, wherein the central server is configured to determine, based on the training log, whether to use the local model provided from the client in generating a new global model.

6. The model generation system according to claim 2, wherein
the client is configured to generate a report related to additional training having been executed and provide the central server with the report, and
the central server is configured to determine, based on the report from the client, whether to use the local model provided from the client in generating a new global model.

7. The model generation system according to claim 6, wherein the central server is configured to change the control information for subsequent additional training at the client in accordance with to a result of the determination based on the report.

8. The model generation system according to claim 6, wherein
the report includes information representing a data distribution of the training data, and
the central server is configured to determine whether to use, in generating a new global model, the local model provided from the client from which the report has been acquired, the determination being performed based on a result of a comparison between: a first data distribution at the client from which the report has been acquired, and a second data distribution resulting from integration of data distributions at clients other than the client.

9. The model generation system according to claim 2, wherein
the training data includes medical information on a patient, and
the client has a one-to-one correspondence with the patient.

10. A model generation method, comprising:
providing a client, by a central server, with a global model to be used in federated learning and control information controlling execution of a trainer at the client;
generating, by the client, a local model by inputting training data to the trainer and causing the trainer to perform training of the global model;
providing the central server with the local model by the client; and
executing, by the central server, control to enable the control information to be changed with a training log of the client.

11. An information processing apparatus, comprising:
at least one memory; and
at least one piece of processing circuitry connected to the memory and configured to
transmit, to a client, a global model to be used in federated learning and control information controlling execution of a trainer at the client,
acquire, from the client, a local model generated by training of the global model with training data by the trainer under control of the control information, and
update the control information in accordance with a training log of the client.

12. An information processing apparatus, comprising:
at least one memory; and
at least one piece of processing circuitry connected to the memory and configured to
acquire, from another information processing apparatus, a global model to be used in federated learning and control information defining a constraint for execution of a trainer to perform training of the global model;
apply, to the trainer, the acquired global model and generate a local model by inputting training data to the trainer under the constraint defined by the control information; and
transmit, to the other information processing apparatus, the local model and information that is related to a training log of the trainer and is correlated with the local model.
